(19) United States
(12) Patent Application Publication
(10) Pub. No.: US 2004/0171011 A1
(43) Pub. Date: Sep. 2, 2004

(54) Immobilized asymmetric double lipid layers

(76) Inventors: Johannes Schmitt, Leipzig (DE); Joachim Noller, Leipzig (DE)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/478,500

(22) PCT Filed: May 14, 2002
(86) PCT No.: PCT/EP02/05268

(30) Foreign Application Priority Data
May 21, 2001 (DE) 101 25 713.9

(51) Int. Cl. C12Q 1/68; B05D 3/00; G01N 33/53; C12M 1/34

(52) U.S. Cl. 435/6; 435/287.2; 427/2.11; 435/7.1

(57) ABSTRACT

The invention relates to a method suitable for the production of immobilized double lipid layers, comprising an inner and an outer lipid layer. The inner and outer lipid layer of said double lipid layers have a defined orientation on the surface of a solid body and are immobilized on the surface of said solid body in a substantially stable and dynamic manner. In order to carry out the inventive method, the surface of a solid body is initially modified in such a way that a surface is formed which preferably interacts with only one of the two lipid layers. In a second step, a double lipid layer is deposited on said modified surface. Preferably, other components, especially protein, are inserted into the double lipid layer.

The invention relates to a process for preparing immobilized lipid double layers (hereinafter called lipid bilayers), wherein the lipid bilayers have a defined orientation as regards the lipid layers on the surface of a solid (so-called solid-supported bilayers). The invention furthermore relates to the use of modified solid so surfaces for preparing such lipid bilayers and to a kit for preparing immobilized lipid bilayers.

As an important constituent of all organisms, natural membranes, i.e. what are termed biomembranes, have been the subject of intensive research for a long time now. In principle, membranes are composed of two layers of lipid molecules, which form a lipid bilayer. This lipid bilayer is hydrophobic internally and hydrophilic toward both outer sides. The structural and functional asymmetry of this lipid bilayer is crucial for the different functions possessed by a membrane. This asymmetry consists, in the first place, in a distribution of various lipid molecules which differs in the two lipid layers (inner leaflet and outer leaflet) which form the lipid bilayer. Various membrane proteins which are located in the biomembrane constitute additional components. These membrane proteins can be transmembrane proteins which span the entire lipid bilayer. Other membrane proteins are only partially embedded in the lipid bilayer for example in the outer lipid layer. In addition, membrane proteins can also be attached to one or both sides of the membrane.

In principle, membrane proteins can move laterally in the lipid matrix. However, the proteins can also be more or less fixed at one defined site by means of specific interactions. In general, it is not possible, however, for membrane proteins to migrate from one lipid layer to the other, which means that the asymmetry which is formed by the different lipids and membrane components is a long-term structural, and hence naturally also functional, property of the membrane.

An example of the asymmetry of biomembranes is what is termed the glycocalix on the outer side of membranes. This glycocalix, which exercises defined recognition and identification functions, is formed by glycoproteins on the outer side of the lipid bilayer and glycolipids in the outer lipid bilayer.

Phosphatidylserine lipids are, for example, typical of the inner lipid layer in an animal cell. During programmed cell death (apoptosis), these particular lipids are also incorporated into the outer lipid layer and, at this site, serve as a recognition pattern for other processes.

Transport proteins, which permit directional transport from the outside toward the inside or from the inside toward the outside represent another very important example of the asymmetry of membranes. In summary, the asymmetry of a biomembrane is the crucial prerequisite for a membrane being able to fulfill its complex tasks.

The properties of biomembranes are of very great interest in the fields of pharmascreening and biosensors, for example. For example, experiments directed toward understanding the very complex transport processes in membranes have been undertaken for a long time now. Furthermore, the receptors which are located in the membrane are naturally also of great importance. For this reason, there is great interest in developing suitable model systems for biomembranes.

However, a problem in this connection has thus far been that the asymmetry, which is crucial for many applications, of the membrane or of the lipid bilayer is as a rule lost when preparing appropriate systems. Thus, ultrasonic appliances or polytrons are frequently used when isolating natural membranes from organisms. Working up of this type results in the formation of vesicular structures whose lipid bilayers are generally orientated randomly. Thus, the lipid layer which is directed outward in the natural system may also point outward in the vesicles. In this case, the vesicles are referred to as being what are termed right-side-out (RSO) vesicles. On the other hand, the inner side of the original membrane can also be directed outward in the resulting vesicles, with the latter then being referred to as being inside-out (ISO) vesicles. As a rule, the vesicles which are formed in connection with such a working-up constitute a mixture of RSO and ISO vesicles. These preparations are not suitable for being used to carry out reliable studies of membrane properties since the vesicles in these preparations are not defined.

A variety of methods are known for analyzing the asymmetry which exists in membranes or the orientation of the two lipid layers. For example, it is possible to analyze the activity of asymmetrically arranged enzymes (Ellman, G. L.; Courtney, A.; Valentino, Jr.; Featherstone, R. M.: Biochem. Pharmacol. 1961 (7), 88) or the release, by means of enzymes, of membrane constituents which are orientated in a particular manner (Warren, L. J. Biol. Chem. 1959 (234), 1971). Another possibility is to label the glycocalix with specifically labeled lectins or to label membrane proteins from protein domains which lie externally or internally with specific antibodies.

Very elaborate methods have been developed for preparing vesicles which have a defined orientation (e.g. Steck, T. L.; Kant, J. A. Methods in Enzymology 1974 (31), 172-180; DePierre, J. W.; Karnovsky, M. L. Journal of Cell Biology 1973 (56), 275-303). These methods made it possible to achieve vesicular structures in which the asymmetry of the natural membrane is retained. However, these vesicles are only suitable under very restricted circumstances for investigating the properties of membranes or the properties of membrane proteins, since it is very difficult to control the size and morphology of the vesicles and, as a result, the system does not have defined and reproducible properties. Furthermore, because of their low degree of stability and difficulty in handling, these vesicular structures are not accessible to automation. It is not possible, therefore, to use vesicles for a screening method which is capable of high throughput, as is required in the field of functional tests and in pharmascreening.

In order to take account of the problem of the poor stability of the vesicular structures and the difficulty of handling them, particularly in automated processes, solid-supported membrane systems have been developed. One possibility in this connection is that of immobilizing single lipid layers, what are termed monolayers, on a solid surface. To achieve this, the surface can first of all, for example, be hydrophobized with alkylsilanes or mercaptans. A lipid layer which is applied on this surface orientates itself with its hydrophobic part directed toward the carrier and the hydrophilic side directed outward. However, this system is not suitable for simulating the natural properties of a membrane since it does not reflect either the dynamics or the molecular arrangement of a natural membrane. In addition to this, the incorporation of transmembrane proteins in such a lipid monolayer is either rendered more difficult or frequently not possible at all.

Immobilizing lipid bilayers on solid surfaces therefore offers a better approach. In order to prepare such solid-supported lipid bilayers, lipid vesicles are fused on a solid surface, with a lipid bilayer being formed spontaneously on the surface in this connection (Tamm, L. K.; McConnell, H. M. Biophysical Journal 47, 105-113 (1985); Salafsky, J.; Groves, J. T.; Boxer, S. Biochemistry 35, 14773-14781 (1996)).

The details of the process of vesicle fusion, and of the formation of the lipid bilayer, are thus far not completely understood. Thus, two possible mechanisms are still being discussed in the literature (Salafsky, J.; Groves, J. T.; Boxer, S. Biochemistry 1996 (35), 14773-14781 and Puu, G.; Gustafson, I. Biochim. Biophys. Acta-Biomembranes 1997 (1327), 149-161). In this connection, the particular question is whether, when fusing with a surface, the vesicle inverts or retains its orientation. However, everything points to the fact that the vesicles orientate themselves randomly during the fusion. The first random event as regards the orientation consequently lies in the step of preparing the membrane vesicles while the second random event lies in the fusion with the solid surface. It has consequently not thus far been possible to control the orientation of the immobilized lipid bilayers.

However, there are various indications that vesicles/cells become affixed on solid surfaces in an orientated manner (Jacobson, Branton Science 1977 (195, 4275), 302-304; Wasserman, B. P.; Hultin, H. O.; Jacobson, B. S. Biotechnol. Bioeng. 1980 (22) 271-287). In this instance, an orientation of the lipid bilayer in which what was originally the inner side of the lipid bilayer is directed outward in the immobilized form was achieved by means of a selectively induced electrostatic interaction of the solid surface with transmembrane proteins (Kalish, D. I.; Cohen, C. M.; Jacobson, B. S.; Branton, D. Biochim. Biophys. Acta 1978 (506), 91-110). Consequently, while it was possible, in this instance, to preserve the asymmetry of the membrane, it was only possible to orientate the inner side of the membrane outward. It was not possible to orientate the membrane with what was originally the outer side facing outward. In addition to this, the enzymic activities decreased as a result of the electrostatic interactions of the membrane with the solid surface, which means that this system is not suitable for functional investigations of the membrane.

The invention therefore sets itself the object of providing a lipid bilayer system which guarantees a defined orientation of the lipid layers on a surface of a solid. This system should be solid-supported so as to ensure that it is stable and easy to handle and also suitable for automating. It should be possible to freely select the orientation, that is the asymmetry, of the lipid bilayer such that what was originally the outer side of the lipid bilayer or the membrane either points outward or else faces the solid surface. The functional and structural properties of the lipid bilayer should also be retained in the immobilized form in order to be able to investigate the natural properties of the lipid bilayer or of the proteins or the like which are present therein, in vitro.

This object is achieved by means of an immobilized lipid bilayer which can be prepared by a process as described in claim 1. Preferred embodiments of this process are implemented in claims 2 to 8. Claims 9 to 13 relate to the use of a modified solid surface for preparing an immobilized lipid bilayer. Claims 14 to 20 relate to an immobilized lipid bilayer or to immobilized proteins, and also to a kit for preparing immobilized lipid bilayers or immobilized proteins. Finally, claims 21 and 22 relate to a process for investigating proteins. The wording of all the claims is hereby incorporated into the description by reference.

The novel process is used to prepare a lipid bilayer which is immobilized on a solid. After the process has been concluded, the lipid bilayer covers the solid as what is essentially a continuous membrane film. The lipid bilayer consists of an inner lipid layer and an outer lipid layer (inner leaflet and outer leaflet, respectively), with these two lipid layers being orientated in a specific manner such that the immobilized lipid bilayer exhibits a defined orientation or asymmetry in regard to the solid surface. The lipid bilayer is immobilized in an essentially stable and dynamic manner on the surface of the solid such that the structural and functional properties of a natural lipid bilayer, in particular of a membrane, are reflected. In accordance with the novel process, the surface of the solid is initially modified in such a way as to form a surface which can preferably interact with one of the two lipid layers, that is with the inner lipid layer or the outer lipid layer. In another step of the process, the lipid bilayer is deposited on this modified solid surface. In this connection, the modification according to the invention of the solid surface enables the lipid bilayer to align itself on the solid surface in such a way that a specific orientation of the lipid bilayer is achieved. According to the invention, it is essentially electrostatic interactions between the modified solid surface and the two layers of the lipid bilayer which are responsible for this, with it being possible for the interactions to be either of an attractive nature or of a repulsive nature. Particularly in combination with the “ultrasoft” surface which is explained in more detail below, the electrostatic interactions lead to the desired orientation of the lipid bilayer on the solid. Further details in this regard ensue from the description which follows and, in particular, from the examples.

The asymmetry of the lipid bilayer can be caused, on the one hand, by different lipids or lipid-analogous molecules being present in the two layers of the lipid bilayer. Furthermore, the asymmetry can also be due to additional components in the lipid layers, with these components being distributed differently in the two lipid layers. Advantageously, these additional components are proteins, in particular membrane proteins. In this connection, the proteins can be what are termed transmembrane membrane proteins which span the entire lipid bilayer in a specific orientation. Examples thereof are various transport proteins which make it possible for directional transport to take place through a membrane or a lipid bilayer. G protein-coupled receptors (GPCRs), which constitute very interesting points of attack for potential drugs, represent an example of such transmembrane proteins. The binding sites of these receptors are located on the outer side of membranes. Consequently, the novel process is particularly well suited for investigating the interaction of these receptors with potential active compounds since, in this case, the orientation of the lipid bilayers which contain these receptors can be regulated such that these binding sites in the immobilized membrane system are accessible from the outside.

Since the novel process can be used to prepare a solid-supported lipid bilayer which is orientated in a specific manner, it is naturally also possible, in this way, to control the orientation of the constituents, that is of the transport proteins, for example, which are integrated in the lipid bilayer. The novel process consequently provides a model membrane system which is outstandingly suitable when investigating and characterizing membrane properties and/or the properties of individual membrane components.

In addition to the transmembrane membrane proteins, which fully or partially span the double layer in a preferred direction, the lipid bilayer can also contain what are termed peripheral components, in particular proteins which are only located in one of the two lipid layers or are located on one of the two surfaces of the lipid bilayer. G protein-coupled receptors (GPR), which also constitute very interesting points of attack for potential drugs, represent an example of these peripheral proteins. They are located on the inner side of the cell membrane and are consequently only accessible after the original orientation has been inverted.

The novel process can be used to immobilize a large number of different lipid structures. In accordance with the process, the lipid structures to be immobilized in this connection are first of all converted into the form of vesicles, which form the solid-supported lipid bilayer according to the invention by means of fusion with the modified solid surfaces. Before being fused with the modified surface, the vesicles are advantageously provided in aqueous dispersion.

Particular preference is given to using vesicles which are composed of natural material such that the lipid bilayer which has been immobilized in accordance with the invention constitutes a model of the natural membrane in which model either what was originally the inner membrane surface or what was originally the outer membrane surface alternately faces outward. Membrane vesicles which have been isolated from erythrocytes or from cells which can be cultured, for example CACO-2 cells, are, for example, particularly suitable for this purpose. In addition, the lipid bilayer can be at least partially constructed from membrane fragments derived from natural cells. The vesicles are prepared using conventional methods. According to the invention, the vesicles are brought into contact with the modified solid surface, resulting in the vesicles spontaneously fusing with each other and forming the lipid bilayer or the membrane layer. The final orientation of the lipid bilayer or the membrane layer can be determined in advance by selecting an appropriately modified surface.

Aside from the plasma membrane of cells, suitable natural membranes also of course include intracellular membranes, which can be derived, for example, from liver or kidney cells. It is possible to use cell organelles, for example, for preparing intracellular membranes. These intracellular membranes are of great interest for various active compound screening applications and/or test applications. Essentially all the enzymes which play a crucial role in drug metabolism are specifically located in the intracellular membranes.

Aside from natural membranes, artificial lipid layers or membranes are also suitable for the novel process. These artificial structures can be assembled (reconstituted) in vitro from different components such that this thereby provides an appropriate membrane vesicle which can be used in the novel process for preparing an immobilized lipid bilayer. Artificial lipid layers, as well as natural lipid layers, can be assembled from different lipids, lipid derivatives and lipid-like and/or lipid-analogous substances. Furthermore, the lipid layers can contain peptides, proteins, nucleic acids, ionic or nonionic surfactants and/or polymers. These additional components of the lipid layers can advantageously be enzymes, receptors and/or ligands which more or less span, or are embedded in, the lipid layer or the lipid bilayer and/or which are attached superficially on the lipid bilayer.

In the novel process, the solid surface is first of all modified in such a way that this thereby controls the subsequent formation of a lipid bilayer, as a result of the fusion of vesicles, as far as its orientation is concerned. In addition, the modification according to the invention of the solid surface provides optimal conditions for the gentle immobilization of a lipid bilayer or a membrane. These optimal conditions relate to a combination of different intermolecular interactions whose superposition results in an attractive force between the lipid bilayer which is to be applied and the solid. At the same time, this prevents any direct contact of the lipid bilayer with the solid. In this way, the characteristic dynamic kinetic properties of the lipid bilayer, in particular the lateral diffusion of the different lipid components, are to a large extent left unchanged.

In a molecular sense, the modification results in the formation of an “ultrasoft” surface which, on the one hand, leaves the lipid bilayer which is immobilized on it to a large extent unaffected and, on the other hand, ensures an adequate distance between the solid surface and the lipid bilayer. This thereby avoids denaturing effects which the solid surface might otherwise exert on the lipid bilayer together with the components which are present in it. In addition, the modification according to the invention of the solid surface has the advantage that interactions of components of the lipid bilayer, in particular of proteins, with the solid surface are to a large extent minimized or eliminated such that the membrane proteins retain their activity, for example, for example their enzyme activity, in the new environment as well. The immobilization according to the invention also preserves the lipid properties of the lipid bilayer, resulting in the natural properties of a membrane being imitated perfectly. Taken overall, the surface modification consequently, among other functions, decouples the lipid bilayer from the solid surface. This is a quite crucial advantage for an application in the field of biosensors, for example, since this field requires immobilized lipid bilayers, in particular membranes, which retain their native properties, that is, for example, enzymic activities and/or channel activities. This advantage of the invention can be particularly advantageously exploited, for example, in an application using biochips.

According to the invention, the choice of a particular modification of the solid surface determines the ultimate orientation of the lipid layers which are immobilized on the surface. For the purpose of depositing the lipid bilayer on the modified surface, it is advantageously possible to prepare membrane vesicles of random orientation, that is what are termed ISO and RSO vesicles, and to bring them into contact with the modified solid. Due to the surface properties of the modified solid, the vesicles essentially only fuse in one particular orientation. As a result, the novel process gives rise to a solid-supported lipid bilayer which possesses a defined orientation.

In another particularly preferred embodiment of the novel process, the modified solid is offered a vesicle preparation which only has one orientation. This can, where appropriate, give rise to larger yields of immobilized lipid bilayer having a specific orientation.

It is possible to use a variety of conventional methods to verify the ultimate orientation of the lipid layers which have been immobilized using the novel process. For example, it is possible to analyze the functionality and/or activity of immobilized membrane proteins which are known to be located on what was originally the inner side, that is, in particular, the cytoplasmic side, or on the outer side, of the membrane or the lipid bilayer. The N-acetylsialic acid which is released by neuramidase can also be investigated. It is also possible to use fluorescence-labeled lectins to label the glycocalix, which is located on the outer side of the original membrane layer. Details of these analytical methods can be obtained from the examples.

Advantageously, the modification according to the invention of the solid surface results in an essentially hydrophilic surface. To achieve this, the surface is first of all fuctionalized. For example, it is possible to apply amino functions, epoxy functions, halogenoalkyl functions and/or thiofunctions to the surface. It is advantageously possible to use silanes for the fuctionalization. Furthermore, preference is also given to mercaptans and/or disulfides, in particular alkyl disulfides. Particular preference is given, for the fuctionalization, to N-(2-aminoethyl)-3-aminopropyltrimethoxysilane (EDA), polyethyleneimine (PEI) and/or cysteamine, in particular cysteamine hydrochloride.

In a further step, these functions are used to adsorb and/or chemisorb interacting molecules. It is also possible to dispense with a prior functionalization of the surface if interaction with other molecules is readily possible due to the material properties of the solid. A variety of polymers, in particular polyelectrolytes, preferably anionic polyelectrolytes, can be used as interacting molecules. Polyampholytes, preferably proteins, and/or polyzwitterions are also suitable according to the invention. Particular preference is given to poly(styrenesulfonate) (PSS), in particular Na poly(styrenesulfonate), and/or poly(styrene-co-maleic anhydride) (PSPMA). Polyoxyethylene bis(glycidyl ether) is a particularly preferred substance which is chemisorbed.

In another preferred embodiment of the novel process, additional interacting molecules, which interact with the interacting molecules which were initially applied, are used for modifying the surface.

In another preferred embodiment, functional substances which can be used for analyzing the properties of the lipid bilayer or of the components which are present in the lipid bilayer are employed as interacting molecules. These functional substances are advantageously dyes, in particular fluorescent dyes, and/or enzymically, chemically and/or photochemically reactive molecules.

According to the invention, the modification of the surface is chosen such that the modified surface essentially only interacts with one of the two lipid layers of the lipid bilayer, with the interaction essentially being of an electrostatic nature, which can be either repulsive or attractive. This thereby ensures that, when the vesicles fuse during the deposition of the lipid bilayer on the solid, the lipid bilayer aligns itself in one specific orientation. In a first embodiment of the invention, the modified surface preferentially interacts with what was originally the inner lipid layer (inner leaflet) of the lipid bilayer. In this connection, the modified surface preferably has a negative net charge. For this purpose, the modification of the solid surface is advantageously performed by preparing “ultrasoft” polymer surfaces having a negative net charge. Orientated vesicle systems, which carry a negative net charge on their outer side, are, for example, employed for depositing the lipid bilayer on this modified solid surface. The modification provides what is essentially a hydrophilic surface which reacts repulsively with the outer lipid layer such that, when a vesicle preparation fuses, the lipid bilayer essentially aligns itself so that what was originally the outer side of the lipid bilayer is directed outward, that is faces away from the solid, and is thus accessible to a variety of investigations.

In a second embodiment of the novel process, the solid surface is modified such that it preferentially interacts with what was originally the outer lipid layer of the lipid bilayer to be immobilized. In this connection, the modified surface preferably has a positive net charge. For this purpose, the modification of the surface is preferably performed by preparing soft polymer surfaces which have a positive net charge. Orientated vesicle systems which carry a negative net charge on the outer side are advantageously employed for depositing the lipid bilayers on this modified solid surface. During fusion, the vesicles then align themselves such that what was originally the outer lipid layer faces the solid. As a result, the components which were originally located in or on the inner side of the lipid bilayer, that is, for example, on the cytoplasmic side, become accessible to investigation. These examples make clear that, depending on the properties of the lipid bilayer to be immobilized, the orientation of the immobilized lipid bilayer on the solid can be controlled by choosing an appropriately suitable surface.

The solid surfaces which have been modified in this way are preferably brought into contact with the vesicular structures of the lipid bilayer to be immobilized within a medium, preferably an aqueous medium, resulting in the vesicles fusing spontaneously with each other and with the modified surface and, in this way, as a result of the properties of the modified surface, forming an immobilized lipid bilayer which exhibits a defined asymmetry. The novel process constitutes a system which is decidedly easy to handle and which only requires slight preparative input.

A variety of conventional support materials can be employed for implementing the novel process. In a preferred embodiment of the invention, an essentially planar support is used as the solid. This support can consist of different materials. Advantageously, the surface can also be prepared from a material which is different from that used for the remainder of the support. The solid surface preferably consists of silicate, a semiconductor, in particular silicon, a precious metal, for example gold, and/or a polymer, for example polystyrene. It is naturally also possible to use other surfaces.

In addition to this, it is possible to use, as surface materials, films which are composed of pulverulent metals, semiconductors, precious metals and/or polymers and which are applied, or which can be applied, to what are to a large extent planar support materials. These support materials can, for example, be substrates made of paper, glass, plastic or the like to which the solids are connected in a suitable manner, in particular by means of bonding or fusing.

Particular preference is given to using dispersible solids which advantageously consist of particles which are, for example, of microscopic dimensions. Materials which are preferred for these solids are aluminates, borates and/or zeolites. Colloidal solutions of precious metals, metals, etc., are also suitable. Very particular preference is given to using silicate particles. These particles can, for example, be conventional silicate spheres.

In a particularly preferred embodiment of the invention, use is made of porous particles, resulting in the surface which is available for the immobilization being enlarged.

In another preferred embodiment, the particles employed are magnetic particles, thereby making it possible to substantially facilitate handling ability in a variety of applications. This is particularly important when using the lipid bilayers which have been immobilized in accordance with the invention in automated processes. Preference is furthermore given to using core plate polymer particles.

The solids which are used are preferably employed in the form of a dispersion. This dispersion is advantageously prepared in aqueous solutions.

The invention further encompasses the use of a modified surface of a solid for preparing an immobilized lipid bilayer which has a defined orientation as regards the lipid layers on the surface of the solid. The modified surface is suitable for immobilizing the lipid bilayer in an essentially stable and dynamic manner. The reader is referred to the above description as regards the other characteristics of using the modified surface.

In addition, the invention encompasses an immobilized lipid bilayer which can be prepared using a process as described above. This immobilized lipid bilayer possesses a defined orientation and is immobilized in an essentially stable and dynamic manner on a solid.

The invention furthermore encompasses immobilized proteins which can be prepared by means of a process as described above. These proteins are proteins which are associated with a lipid bilayer which, for its part, is immobilized on a solid. The proteins are, in particular, membrane proteins which span the lipid bilayer entirely or partially, which span, or are embedded in, only one of the lipid layers forming the lipid bilayer, or which are embedded or attached on one or both sides of the lipid bilayer.

The invention also encompasses a kit for preparing immobilized lipid bilayers. This kit is envisaged for immobilizing lipid bilayers such that these lipid bilayers exhibit, with their inner lipid layer and their outer lipid layer, a defined orientation on the surface of a solid. The orientation can be determined in advance. The lipid bilayers which are immobilized in this way are fixed on the solid in what is essentially a stable and dynamic manner such that they are particularly suitable for functional and structural investigations of the lipid bilayer, for example of a membrane. This kit comprises at least one solid having a modified surface which is suitable for immobilizing the lipid bilayer in the sense of the invention. The reader is referred to the above description in regard to the properties of this modified solid surface. It may furthermore be preferred for the kit not to contain the finally modified surface but, instead, the various components which are required for preparing such a modified surface. This may be advantageous, for example, for storage and/or stability reasons. In another preferred embodiment of the novel kit, the latter additionally contains lipid vesicles, with these vesicles preferably exhibiting additional components, in particular proteins. These lipid vesicles are envisaged for fusing on the modified solid surface and therefore constitute the material for the immobilized lipid bilayer. In this connection, it may be preferable for the user himself to provide additional components for the lipid bilayer and to introduce these components into the lipid vesicles. The lipid vesicles are advantageously in the form of a dispersion. This can, for example, be an aqueous dispersion which may possibly contain appropriate additives which improve the stability and durability of the lipid vesicles. The lipids can naturally also be provided in a form other than vesicles, for example in dry form.

The invention further encompasses a kit for preparing immobilized proteins which are located in or on lipid bilayers which are for their part immobilized in a defined orientation. This kit comprises at least one solid having a modified surface and, preferably, lipids, in particular in the form of vesicles, which exhibit the proteins to be immobilized. These proteins are preferably membrane proteins which find their natural environment as a result of being immobilized in lipid bilayers and can thus be analyzed under what are virtually native conditions. The reader is referred to the above description concerning the various details in regard to the immobilized proteins.

Finally, the invention encompasses a process for investigating proteins, in particular membrane proteins, with these proteins being employed as immobilized proteins in lipid bilayers which are for their part immobilized. The above description is likewise referred to in regard to the proteins which are employed and which are immobilized in lipid bilayers. The further implementation of the process is undertaken using customary methods which are familiar to a skilled person in this field.

The features which have been described, and also other features of the invention, ensue from the figures and the following description of examples taken in combination with the subclaims. In this connection, the different features can in each case be realized either on their own or in combination with each other.

In the figures:

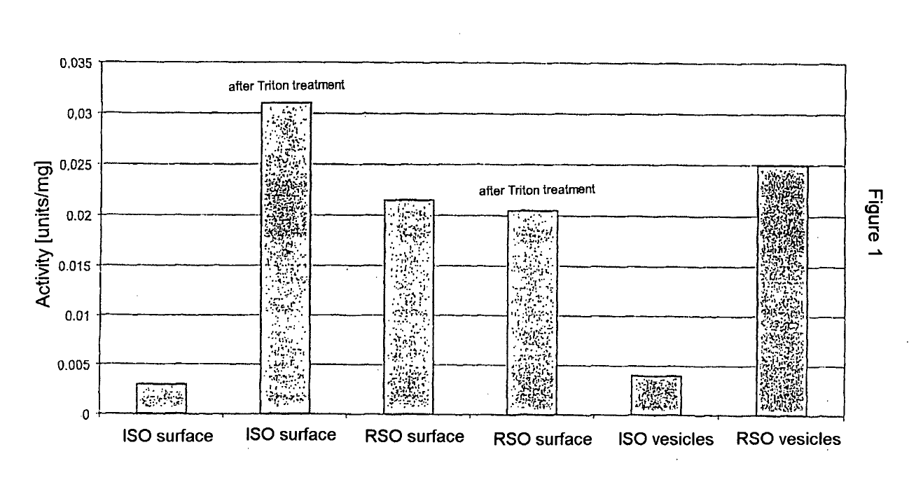

FIG. 1 shows the use of acetylcholinesterase activity for comparing erythrocyte membranes which have been immobilized in accordance with the invention in different orientations on the solid surface,

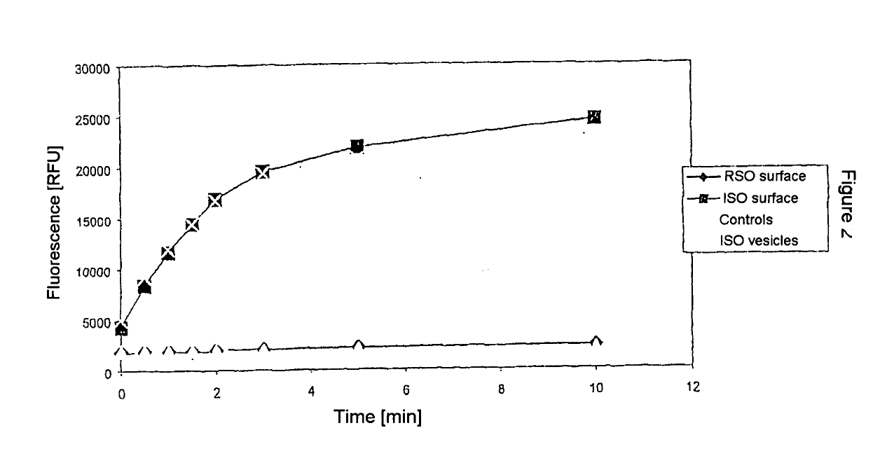

FIG. 2 shows the use of the hydrolytic activity of the P glycoprotein in the presence of a modulator (verapamil) for comparing CACO-2 plasma membranes which have been immobilized in accordance with the invention in different orientations on the solid surface,

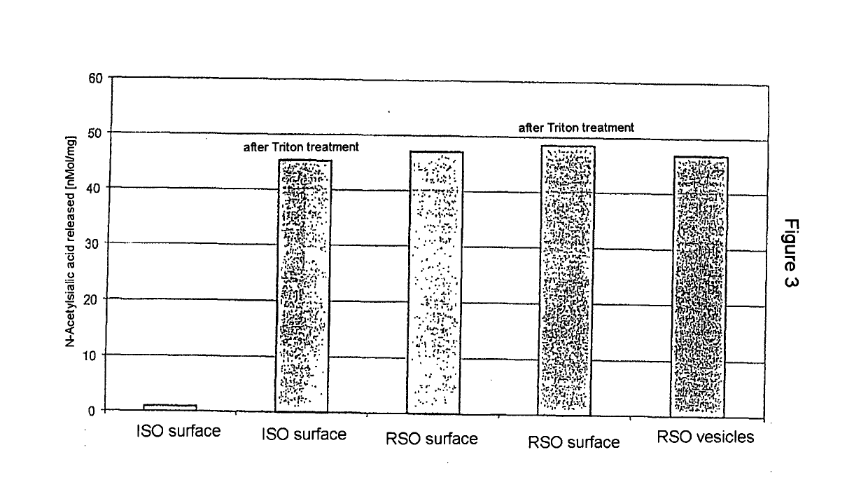

FIG. 3 shows the use of neuramidase-released N-acetylsialic acid for comparing plasma membranes on HEK293 cells, which membranes have been immobilized in accordance with the invention, in different orientations on the solid surface, and

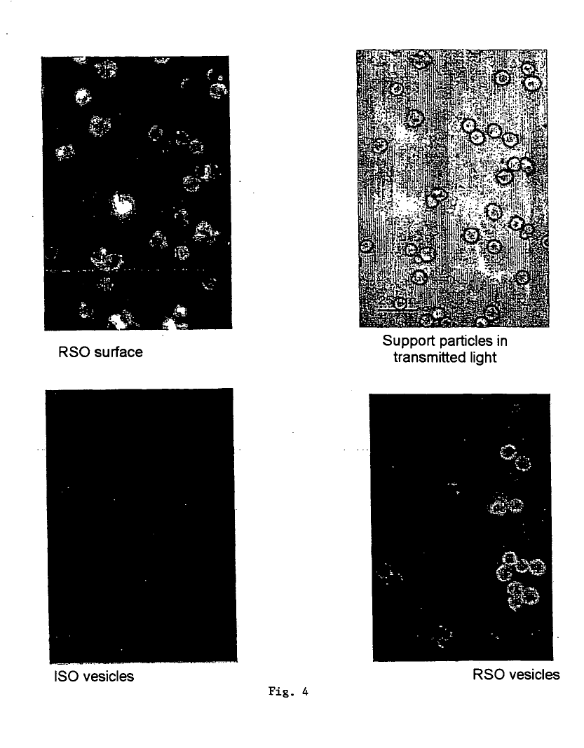

FIG. 4 shows the use of the binding of peanuts lectin-FITC to the glycocalix, following neuramidase treatment, for comparing erythrocyte membranes which have been immobilized in accordance with the invention in different orientations on the solid surface.

### EXAMPLES

### 1. Modifying the Solid Surface

1.1. Functionalizing the Solid Surface

1.1.1 Amino-functionalizing Pulverulent and Porous Silicate Surfaces Using N-(2-aminoethyl)-3-amino-propyltrimethoxysilane (EDA)

A silane solution, consisting of 9.2 ml of N-(2-aminoethyl)-3-aminopropyltrimethoxysilane (EDA) and 243 μl of concentrated acetic acid in 450 ml of deionized water, was prepared fresh. After 5 minutes, 2 g of a porous silicate material (Nucleosil 50-10 from Machery-Nagel, Duren) were added to the silane solution and suspended by shaking. This dispersion was rotated slowly for three hours and, after that, the silicate material is sedimented and washed three times with deionized water. The success of the silanization is documented by means of diffuse reflectance infrared spectroscopy (DRIFT), which is performed on the dried silicate material. Other support materials having pore sizes between 5 and 400 nm were functionalized in a similar manner.

1.1.2.Amino-functionalizing Pulverulent and Porous Silicate Surfaces Using Polyethyleneimine (PEI)

5 g of a porous silicate material (Nucleosil 4000-10) from Machery-Nagel, Düren) were added to a polyethyleneimine (PEI) solution consisting of 250 mg of PEI (50% solution in water, Aldrich, Steinheim) in 50 ml of deionized water, and the mixture was slowly rotated for three hours. After that, the silicate material was sedimented and washed three times with deionized water. The success of the reaction was documented by means of diffuse reflectance infrared spectroscopy (DRIFT), which was performed on the dried silicate material. Other support materials having pore sizes of between 5 and 400 nm were functionalized in a similar manner.

1.2. Selecting Particularly Preferred Properties

1.2.1.Chemisorbing Polyoxyethylene Bis(glycidyl Ether) (PEG-DiGly) on EDA-functionalized Silicate Surfaces

1 g of a silicate material which had been amino-functionalized as described in Example 1.1.1 was added to 10 ml of a 5% solution consisting of polyoxyethylene bis(glycidyl ether) (PEG-DiGly) (Mw˜3350 SIGMA) in phosphate buffer (pH 7.4), and the mixture was shaken for three hours. After that, the silicate material was sedimented and washed three times with deionized water. The success of the chemisorption was documented by means of DRIFT.

1.2.2 Adsorbing Na Poly(styrenesulfonate) (PSS) on Polyethyleneimine-functionalized Silicate Surfaces

1 g of a silicate material which had been amino-functionalized as described in Example 1.1.2. was added to an Na poly(styrenesulfonate) (PSS) solution consisting of 25 mg of PSS (Mw˜70,000, Aldrich, Steinheim) in 50 ml of a 3-molar NaCl solution, and the mixture was shaken for three hours. After that, the silicate material was sedimented and washed three times with deionized water. The success of the adsorption was documented by means of DRIFT and the decrease in the concentration of PSS in the solution was also documented.

### 2. Depositing Lipid Layers on the Modified Surfaces

2.1. Immobilizing Native Erythrocyte Membranes on Untreated Silicate Surfaces and Measuring the Acetylcholinesterase Function

Orientated RSO and ISO plasma membrane vesicles were prepared using a method developed by Steck and Kant (Methods in Enzym. 1974, 31, 172-180). Extrusion was then used to convert this dispersion into small, single-shell vesicles having a diameter of 20-90 nm. 50 mg of a porous silicate support (Machery-Nagel, Düren) were added to in each case 90 μl of the solutions (about 0.5 mg of total protein) and the mixtures were incubated at 4° C. for 18 hours, with 100 mM triethanolamine (pH 7.4) and 100 mM NaCl being used as the buffer solution (incubation buffer). After that, the support material was sedimented and washed three times with incubation buffer. The success of the coating was documented by means of DRIFT, which was performed on the dried material. The activity of the acetylcholinesterase, which was present in the outer layer, was determined, using a method developed by Ellman et al. (Biochem. Pharmacol. 1961, 7, 88), on the support material after the washing in the incubation buffer. In this connection, it is important to use detergent in a control to produce holes in the lipid bilayer and, in this way, to measure any enzyme molecules which may possibly be facing away from the surface (FIG. 1). These function tests verified that an inversion took place in connection with the fusion with the solid surface which was offered, and also verified the uniformity (>90%) of the surface which was obtained.

2.2. Immobilizing Native CACO-2 Membranes on Different Silicate Surfaces and Measuring the ATPase Function of the P Glycoprotein

ISO-orientated plasma membrane vesicles derived from CACO-2 cells were prepared using a method developed by Schlemmer and Sirotnak (Anal. Biochem. 1995, 228, 226-231). Extrusion was then used to convert this dispersion into small, single-shell vesicles having a diameter of 20-90 nm. In each case 50 mg of a porous silicate support which had been modified as described in Example 1.2.1. or Example 1.2.2. were added to 900 μl of this solution (about 0.5 mg of total protein) and the mixtures were incubated at 4° C. for 18 hours using 100 mM triethanolamine (pH 7.4) and 100 mM NaCl as the buffer solution (incubation buffer). After that, the support materials were sedimented and washed three times with incubation buffer. The success of the coating was documented by means of DRIFT, which was performed on the dried material. The activity of the P glycoprotein on the support materials was determined, after the washing in the incubation buffer, by measuring the ATP hydrolysis activity in dependence on a known modulator (verapamil). In the case of the support which was modified as described in Example 1.2.2., an RSO-orientated surface is obtained after the immobilization, with this surface not possessing any significant enzyme activity, however, due to the incorrect orientation of the protein to be investigated. The situation was different with the ISO surface which was prepared using the support described in Example 1.2.1. This surface possesses a hydrolysis activity (FIG. 2) on the support material which is comparable to that of the isolated vesicles.

2.3. Immobilizing Native HEK293 Membranes on Different Silicate Surfaces and Measuring the N-acetylsialic Acid Released by Neuramidase

RSO-orientated plasma membrane vesicles derived from HEK293 cells were prepared using a method developed by DePierre and Karnovsky (Journal of Cell Biology 1973, 56, 275-303). Extrusion was then used to convert this dispersion into small, single-shell vesicles having a diameter of 20-90 nm. In each case 50 mg of a porous silicate support which had been modified as described in Example 1.2.1. or Example 1.2.2. were added to 900 μl of this solution (about 0.5 mg of total protein) and the mixtures were incubated at 4° C. for 18 hours using 100 mM triethanolamine (pH 7.4) and 100 mM NaCl as the buffer solution (incubation buffer). After that, the support materials were sedimented and washed three times with incubation buffer. The success of the coating was documented by means of DRIFT, which was performed on the dried material. A kit supplied by CalBiochem (Bad Soden) was used, in accordance with the enclosed protocol, to measure the release of Nacetylsialic acid by neuramidase on the support materials after the washing in the incubation buffer. In the case of the support which was modified as described in Example 1.2.1., an ISO-orientated surface is obtained after the immobilization, with this surface not, however, exhibiting any significant release of Nacetylsialic acid by neuramidase due to the incorrect orientation of the glycocalix to be investigated. A comparable release can only be achieved by adding detergent. The situation is different in the case of the RSO surface which was prepared using the support described in Example 1.2.2. This surface exhibits a release on the support material which is comparable to that in the case of the isolated vesicles (FIG. 3).

2.4. Immobilizing Native Erythrocyte Membranes on PEI/PSS-modified Silicate Surfaces and Labeling the Glycocalix with FITC-lectins

Orientated RSO and ISO plasma membrane vesicles were prepared using a method developed by Steck and Kant (Methods in Enzym. 1974, 31, 172-180). Extrusion was then used to convert these dispersions into small, single-shell vesicles having a diameter of 20-90 nm. 50 mg of a porous silicate support which had been modified as described in Example 1.2.2. were added to 900 μl of a 1:1 (v/v) mixture of these solutions (in all about 0.5 mg of total protein), and the mixture was incubated at 4° C. for 18 hours using 100 mM triethanolamine (pH 7.4) and 100 mM NaCl as the buffer solution (incubation buffer). After that, the support material was sedimented and washed three times with incubation buffer. The success of the coating was documented by means of DRIFT, which was performed on the dried material. The glycocalix (outer side of the RSO vesicles) was labeled by means of binding peanut lectin (FITC-coupled, Sigma-Aldrich GmbH, Munich) on the support material after the washing in the incubation buffer, with this labeling being documented in a fluorescence microscope. This labeling verified that only an RSO surface, with a uniformity of at least 90%, was produced (FIG. 4).

### 3. Properties of the Membranes on Optimized Support Material

3.1. Stability in a Flowing Aqueous Medium

The systems described in Examples 2.1. to 2.4. were exposed to a flowing medium (incubation buffer of the respective example) in a test bath for a period of 24 hours. In each case equal quantities of support material were removed from the test bath at intervals of 2 hours and dried; DRIFT was then used to analyze the coating of this support material. No measurable decrease in the membrane coating with time was observed.

3.2. Stability Following Freezing

The systems described in Examples 2.1. to 2.4. were frozen in a dispersed state at −800° C. and then brought to room temperature and dried. Comparative DRIFT measurements performed before and after the freezing showed that the quantities of lipid and protein on the support material were unchanged. In addition, the described systems were stored, following preparation, for a period of 3 months at −800° C. Samples were removed at intervals of 1 month and analyzed in regard to measurable variables described in the examples. After 2 months, the enzyme activities had fallen to approx. 70% of the original value (as measured immediately after preparing and washing the support material) whereas the results for the labeling of the glycocalix with FITC-lectin were unaltered. It was not possible to detect any protein in the respective supernatants from the stored samples.

1. A process for preparing an immobilized lipid bilayer having an inner lipid layer and an outer lipid layer, with the lipid bilayer exhibiting a defined orientation in regard to the lipid layers on a surface of a solid and being immobilized in an essentially stable and dynamic manner on the surface of the solid, comprising the following process steps:
a) modifying the surface of the solid in order to form a surface which preferably interacts with only one of the two lipid layers, and
b) depositing the lipid bilayer on the modified surface.

2. The process as claimed in claim 1, characterized in that additional components, in particular proteins, are inserted into the lipid bilayer.

3. The process as claimed in claim 1 or claim 2, characterized in that the deposition of the lipid bilayer is performed by fusing lipid vesicles.

4. The process as claimed in one of the preceding claims, characterized in that the modification of the surface of the solid comprises the following constituent steps:
aa) functionalizing the surface and/or
ab) adsorbing and/or chemisorbing interacting molecules.

5. The process as claimed in one of the preceding claims, characterized in that the surface which has been modified in accordance with process step
a) interacts with the inner lipid layer.

6. The process as claimed in claim 4 or claim 5, characterized in that the modification of the surface is performed by applying polymer films having a negative net charge.

7. The process as claimed in one of claims 1 to 4, characterized in that the surface which has been modified in accordance with process step a) interacts with the outer lipid layer.

8. The process as claimed in claim 7, characterized in that the modification of the surface is performed by applying polymer films having a positive net charge.

9. The use of a modified surface of a solid for preparing an immobilized lipid bilayer having an inner lipid layer and an outer lipid layer, with the lipid bilayer exhibiting a defined orientation in regard to the lipid layers on a surface of a solid and being immobilized in an essentially stable and dynamic manner on the surface of the solid.

10. The use as claimed in claim 9, characterized in that the surface of the solid is envisaged for interacting with the inner lipid layer.

11. The use as claimed in claim 10, characterized in that the modified surface exhibits a negative net charge, in particular a polymer film having a negative net charge.

12. The use as claimed in claim 9, characterized in that the surface of the solid is envisaged for interacting with the outer lipid layer.

13. The use as claimed in claim 12, characterized in that the modified surface exhibits a positive net charge, in particular a polymer film having a positive net charge.

14. An immobilized lipid bilayer which can be prepared by a process as claimed in one of claims 1 to 9.

15. An immobilized protein, in particular a membrane protein, which can be prepared by a process as claimed in one of claims 2 to 8.

16. A kit for preparing immobilized lipid bilayers having inner and outer lipid layers, with the lipid bilayers exhibiting a defined orientation in regard to the lipid layers on the surface of solids and being immobilized in an essentially stable and dynamic manner on the surface of the solids, at least comprising a solid having a modified surface which preferably interacts with only one of the two lipid layers.

17. A kit as claimed in claim 16, which furthermore comprises lipid vesicles which preferably exhibit additional components, in particular proteins.

18. A kit for preparing immobilized proteins in immobilized lipid bilayers having inner and outer lipid layers, with the lipid bilayers exhibiting a defined orientation in regard to the lipid layers on the surface of solids and being stabilized in an essentially stable and dynamic manner on the surface of the solids, at least comprising a solid having a modified surface, which preferably interacts with only one of the two lipid layers, and also, preferably, lipid vesicles which exhibit proteins.

19. A kit as claimed in one of claims 16 to 18, characterized in that the modified surface can be prepared using a process as claimed in claim 5 or claim 6.

20. A kit as claimed in one of claims 16 to 18, characterized in that the modified surface can be prepared using a process as claimed in claim 7 or claim 8.

21. A process for investigating proteins, in particular membrane proteins, characterized in that these proteins are employed as immobilized proteins in immobilized lipid bilayers having inner and outer lipid layers, with the lipid bilayers exhibiting a defined orientation in regard to the lipid layers on the surface of solids and being immobilized in an essentially stable and dynamic manner on the surface of the solids.

22. The process as claimed in claim 21, characterized in that the immobilized lipid bilayers can be prepared using a process as claimed in one of claims 1 to 8.
